(19) United States
(12) Patent Application Publication
(10) Pub. No.: US 2025/0099273 A1
(43) Pub. Date: Mar. 27, 2025

(54) STENT FORMED OF WIRE FRAMEWORK

(71) Applicant: BOSTON SCIENTIFIC SCIMED, INC., Maple Grove, MN (US)

(72) Inventors: LOUIS MCNERN, Donegal (IE); GARY GILMARTIN, Foxford (IE); ENDA CONNAUGHTON, Galway (IE)

(73) Assignee: BOSTON SCIENTIFIC SCIMED, INC., Maple Grove, MN (US)

(21) Appl. No.: 18/894,775

(22) Filed: Sep. 24, 2024

Related U.S. Application Data
(60) Provisional application No. 63/585,107, filed on Sep. 25, 2023.

(51) Int. Cl. A61F 2/89

(52) U.S. Cl. CPC A61F 2/89; A61F 2220/0025; A61F 2250/0037

(57) ABSTRACT

Devices, systems, and methods for treating defects in anatomical structures are disclosed. A device may include a stent having a tubular member with a first end, a second end, and a lumen between the first end and the second end. A wire having a first end and a second end may form a plurality of annular rows between the first end and the second end, where the rows may define the lumen. The wire of the stent may define a plurality of turns in each row of the plurality of annular rows and may have no overlapping portions along the rows. A gap may extend between consecutive rows of the plurality of rows and a connecting segment may span across the gap to interconnect two rows of the plurality of rows. The connecting segment may be the only portion of the wire that spans across the gap.

### CROSS REFERENCE TO RELATED APPLICATIONS

The present application claims the benefit of and priority to U.S. Provisional Patent Application Ser. No. 63/585,107, filed on Sep. 25, 2023, the disclosure of which is incorporated herein by reference.

### TECHNICAL FIELD

The present disclosure pertains generally, but not by way of limitation, to medical devices and systems, and methods of treatment. More particularly, the present disclosure relates to stents, stent configurations, and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure. These devices are manufactured by any one of a variety of different manufacturing methods and may be used according to any one of a variety of methods for a variety of applications. Of the known medical devices, delivery systems, and methods, each has certain advantages and disadvantages. For example, in some stents, the compressible and flexible properties that assist in stent delivery may also result in a stent that has a tendency to migrate from its originally deployed position in a body lumen. There is an ongoing need to provide alternative medical devices and delivery devices as well as alternative methods for manufacturing and using medical devices and delivery devices, such as those susceptible to migration in the anatomy.

### BRIEF SUMMARY

This disclosure provides design, material, manufacturing method, and use alternatives for medical devices.

In a first example, a stent comprises a tubular member having a first end, a second end, and a lumen between the first end and the second end, wherein a wire having a first end and a second end may form a plurality of annular rows between the first end and the second end that define the lumen, wherein the wire may have no overlapping portions along the plurality of rows, wherein the wire may form a plurality of turns in each row of the plurality of rows, wherein a gap may extend between consecutive rows of the plurality of rows, and wherein the wire may include a connecting segment that spans across the gap to interconnect two rows of the plurality of rows.

Additionally or alternatively to any of the examples above, the connecting segment may be the only portion of the wire that spans across the gap.

Additionally or alternatively to any of the examples above, the connecting segment may be at an oblique angle relative to a longitudinal axis of the plurality of rows.

Additionally or alternatively to any of the examples above, a first row of the

consecutive rows may have a first height and a second row of the consecutive rows has a second height, and the connecting segment may have a length greater than a sum of the first height and the second height.

Additionally or alternatively to any of the examples above, the plurality of turns in each row define peaks at a first axial end of a row and valleys at a second axial end of a row, and the gap circumferentially extends between all of the peaks of a first row of the consecutive rows and all of the valleys of a second row of the consecutive rows.

Additionally or alternatively to any of the examples above, the plurality of turns in each row may define peaks at a first axial end of a row and valleys at a second axial end of a row, and a height between the peaks and valleys of at least one row of the plurality of rows may be centered about an axial location along a length of the plurality of rows.

Additionally or alternatively to any of the examples above, the plurality of turns may form a pattern that repeats in a row of the plurality of rows and the pattern may comprise three turns with each of the three turns at a different axial location of the row of the plurality of rows.

Additionally or alternatively to any of the examples above, the plurality of turns may form a pattern that repeats in a row of the plurality of rows and the pattern may comprise two turns with each of the two turns at a same axial location of the row of the plurality of rows.

Additionally or alternatively to any of the examples above, the two turns at the same axial location may be independent of turns of an adjacent instance of the pattern.

Additionally or alternatively to any of the examples above, a coating is applied to the plurality of rows.

In a further example, a stent may comprise a wire defining a tubular member with a lumen, the wire having a first end and a second end, and wherein the wire may define a plurality of annular rows between the first end and the second end, and wherein each row may have a pattern that repeats and the pattern includes three or more turns with each of the three or more turns at a different axial location along a length of the plurality of rows and two or more turns with each of the two or more turns at a same axial location along the length.

Additionally or alternatively to any of the examples above, the two or more turns at the same axial location along the length may be independent of turns in an adjacent instance of the pattern.

Additionally or alternatively to any of the examples above, the wire may have no segments that overlap other segments of the wire.

Additionally or alternatively to any of the examples above, each row may be centered about an axial location along the length of the plurality of rows.

Additionally or alternatively to any of the examples above, a single connecting segment of the wire connects consecutive rows of the plurality of rows.

In a further example, a stent may comprise a wire defining a tubular member with a lumen, the wire having a first end and a second end, and wherein the wire defines a plurality of annular rows between the first end and the second end, and wherein each row has a pattern that repeats circumferentially and the pattern includes two or more turns each at a same axial location along a length of the plurality of rows and all turns of the pattern are independent of all turns in an adjacent instance of the pattern.

Additionally or alternatively to any of the examples above, the pattern may include three or more turns each at a different axial location along the length of the plurality of rows.

Additionally or alternatively to any of the examples above, the wire may have no segments that overlap other segments of the wire.

Additionally or alternatively to any of the examples above, each row may be centered about an axial location along the length of the plurality of rows.

Additionally or alternatively to any of the examples above, a single connecting segment of the wire may connect consecutive rows of the plurality of rows.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic overview of a biliary and/or pancreatic tree;

FIG. 2 is a schematic view of an illustrative stent device;

FIG. 3 is a schematic view of an illustrative stent device, where the stent device is laid flat;

FIG. 4 is a schematic view of a section of framework having an illustrative configuration of a wire pattern for a stent device, with the stent device in an unconstrained configuration;

FIG. 5 is a schematic view of the section of framework having the illustrative configuration of a wire pattern for a stent depicted in FIG. 3, with the stent device in a constrained configuration;

FIG. 6 is a schematic view of an illustrative wire pattern for a stent device;

FIG. 7 is a schematic view of an illustrative stent device with a coating;

FIG. 8 is a schematic view of an illustrative configuration of a wire for a stent device, with a coating applied to the wire;

FIG. 9 is a schematic view of a section of framework having an illustrative configuration of rows for a stent device; and

FIG. 10 is a schematic view of a section of framework having an illustrative configuration of rows for a stent device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Endoscopic retrograde cholangiopancreatography (ERCP) is primarily used to diagnose and treat conditions of the bile ducts, including, for example, gallstones, inflammatory strictures, leaks (e.g., from trauma, surgery, etc.), and cancer. Through the endoscope, the physician can see the inside of the stomach and the duodenum, and inject dies into the ducts in the bile tree and pancreas so they can be seen on X-rays. These procedures may necessitate gaining and keeping access to the biliary duct, which may be technically challenging, may require extensive training and practice to gain proficiency, and may require one or more expensive tools in order to perform. Blockage of the biliary duct may occur in many of the disorders of the biliary system, including the disorders of the liver, such as, primary schlerosing cholangitis, stone formation, scarring in the duct, etc. This requires the need to drain blocked fluids from the biliary system, to treat the disorders.

During an ERCP procedure, a number of steps are typically performed while the patient is often sedated and anaesthetized. For example, an endoscope may be inserted through the mouth, down the esophagus, into the stomach, through the pylorus into the duodenum, to a position at or near the ampulla of Vater (the opening of the common bile duct and pancreatic duct). Due to the shape of the ampulla and the angle at which the common bile and pancreatic ducts meet the wall of the duodenum, the distal end of the endoscope is generally placed just past the ampulla. Due to positioning of the endoscope beyond the ampulla, the endoscopes used in these procedures are usually side-viewing endoscopes. The side-viewing feature provides imaging along the lateral aspect of the tip rather than from the end of the endoscope. This allows the clinician to obtain an image of the medical wall of the duodenum, where the ampulla of Vater is located, even though the distal tip of the endoscope is beyond the opening.

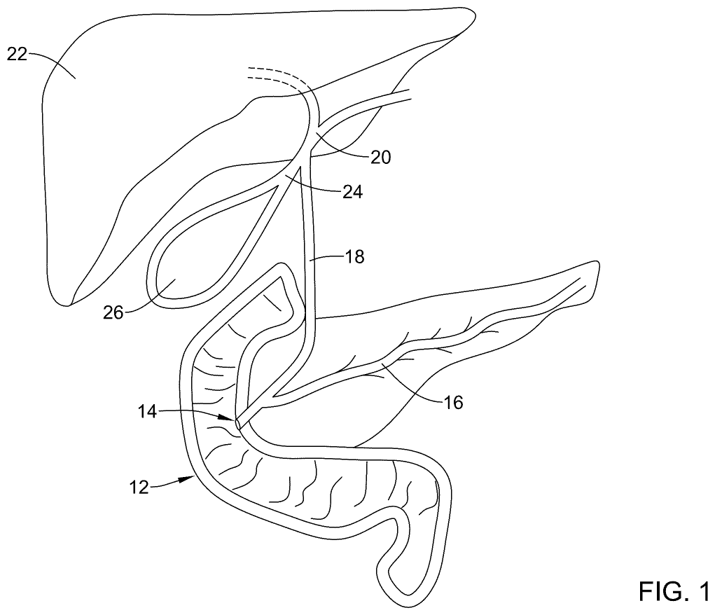

FIG. 1 illustrates an overview of the biliary system or tree. The ampulla of Vater 14 is located at the illustrated portion of the duodenum 12. For the purpose of this disclosure, the ampulla of Vater 14 is understood to be of the same anatomical structure as the papilla of Vater. The ampulla of Vater 14 generally forms the opening where the pancreatic duct 16 and the bile duct 18 can empty into the duodenum 12. The hepatic ducts, denoted by the reference numeral 20, are connected to the liver 22 and empty into the bile duct 18. Similarly, the cystic duct 24, being connected to the gall bladder 26, also empties into the bile duct 18. In general, an endoscopic or biliary procedure may include advancing a medical device to a suitable location along the biliary tree and then performing the appropriate intervention.

Accessing a target along the biliary tree may often involve advancing an endoscope through the duodenum 12 to a position adjacent to the ampulla of Vater 14, and advancing a medical device, which may be a stent, through the endoscope and through the ampulla of Vater 14 to the intended target site. The intended target site may be, for example, the common bile duct 18 and/or the pancreatic duct 16.

Applying a stent to a duct of the biliary tree may reduce obstructions and enable the duct (e.g., a bile duct and/or other suitable duct) to remain patent (e.g., open) in a presence of a stricture. When the stent is deployed from a delivery catheter, the stent radially expands and keeps the lumen patent, which may facilitate bile drainage through the duct.

Stent deployment may be effected in any suitable manner. In some examples, stent deployment may include delivering a stent in a distal end of a delivery system (e.g., a co-axial delivery system and/or other suitable delivery system) to a target location or site within a patient (e.g., at a location of a biliary stricture and/or other suitable location), positioning a proximal handle of a delivery device against a chest or stomach of a practitioner (e.g., a physician, nurse, etc.), and pulling on a distal handle in a proximal direction towards the proximal handle. Pulling the distal handle in the proximal direction may slide a sheath (e.g., any suitable external tube, which may be known as an e-tube) covering the stent proximally to expose the stent while maintaining a position of an inner elongate member at the target location or site. As the sheath is withdrawn from the stent, the stent radially expands and shortens lengthwise (e.g., the stent foreshortens in a proximal direction). As a result of the shortening of the stent, the practitioner must consider an expected shortening (e.g., shortening in the proximal direction and/or other shortening) of the stent when positioning the stent and delivery device at the target location or site, which may result in poor alignment of the stent with the target location or site (e.g., relative to a location of a target stricture, etc.) and/or needing to re-position the stent after initial deployment.

Shortening of a stent during deployment may occur with stents having braided, knitted or overlapping structure. Stents formed by laser cutting a sheet of material may be less prone to shortening upon deployment than stents having a braided, knitted, or overlapping structure and as such, may provide practitioners with increased control over positioning of a stent across the target location or site relative to the control provided when using a braided, a knitting, or overlapping structure. Further, stents of a laser cut construction may have a lower constrained diameter (e.g., diameter when in the delivery device) relative to a constrained diameter of braided, knitted, or overlapping stents, which may facilitate delivering the stent to small diameter ducts, such as hepatic and/or other biliary ducts, using a small diameter delivery device (e.g., having a 6 F diameter and/or other suitable diameter). In some cases, such a lower constrained diameter and a small diameter delivery device may facilitate dual stenting of the hepatic and/or other biliary ducts, where two delivery devices are placed through an endoscope to a target location or site and are used to deploy the stents simultaneously.

However, stents having a laser cut construction have drawbacks. For example, stents of a laser cut construction are often bare or uncovered, which results in tissue ingrowth at and/or around the stent that makes removal of the stent after a period of time difficult or impossible without injuring the patient. In another example, stents having a laser cut construction cannot be re-constrained after at least partial deployment during placement, which may complicate positioning the stent at the target location or site.

The stent configurations discussed herein may be configured to have a small constrained diameter and mitigate foreshortening during deployment of the stent. Additionally, the stent configurations discussed herein may be configured 1) to facilitate being re-constrained after at least partial deployment and 2) to be covered and/or coated to prevent or mitigate tissue ingrowth after initial deployment. In one example, the stent configurations discussed herein may be formed from one or more wires having parallel or non-overlapping wire segments both in a constrained, delivery configuration, and an expanded, deployed configuration, which facilitate mitigating foreshortening of the stent and the stent having a small, constrained diameter relative to other configurations of wire stents (e.g., braided, knitted, or overlapping wire stents).

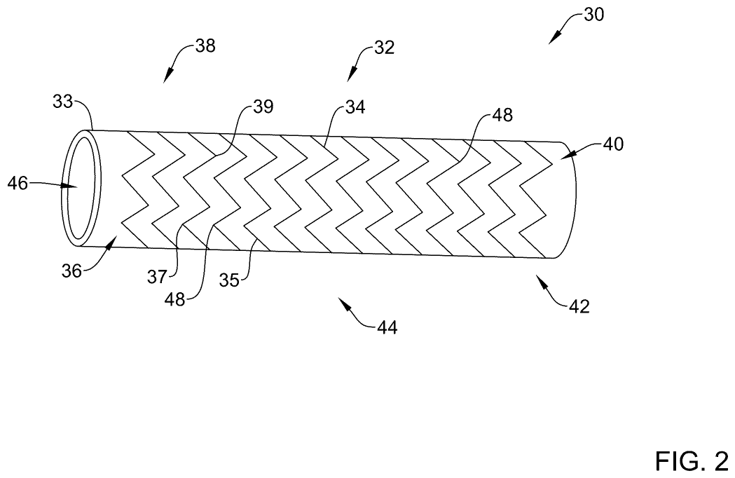

FIG. 2 depicts a schematic view of a stent 30 having a tubular member 32. As seen in FIG. 2, the tubular member 32 includes a tubular framework 34 (e.g., an expandable framework) extending along a length of the stent 30, which may be formed from one or more wires or filaments, and/or other suitable components. The framework 34 may include a number of segments 35 of the wire(s) to form a structure of the framework 34. The segments 35 may converge at apices as the wire(s) extend circumferentially around the tubular member 32, such as in a zig-zag configuration. In some examples, the stent 30 may be formed from a single wire defining the segments 35 of the framework 34 with the single wire extending the entire length of the tubular framework 34. As shown in FIG. 2, the stent may include a covering 33, such as a polymeric coating or sleeve, disposed on the tubular framework 34 and extending along the entire length of the stent 30, or a portion thereof. In other instances, the stent 30 may be devoid of the covering 33.

The framework 34 may extend from a proximal end 36 within a proximal end region 38 to a distal end 40 within a distal end region 42, with an intermediate region 44 extending between the proximal end region 38 and the distal end region 42. In some examples, the framework 34 may define a lumen 46 extending through the tubular member 32 from the proximal end 36 to the distal end 40.

The segments 35 of the framework 34 may be adapted to transition from a radially compressed or constrained configuration (e.g., a delivery configuration) to a radially expanded configuration (e.g., a deployed configuration), for example. In some instances, the segments 35 may be arranged in a suitable pattern, such as a serpentine or sinusoidal configuration and/or other suitable arrangement. For example, the wire forming each of the segments 35 may extend in an undulating pattern to form a number of alternating peaks 37 at a first end (e.g., a first axial end) of the annular row 56 where segments 35 converge at the turns 48 (e.g., pointing to the proximal end 36) and valleys 39 at a second end (e.g., a second axial end) of the annular row 56 where segments 35 converge (e.g., pointing to the distal end 40) at the turns 48. The turns 48 are considered the point along a wire where the wire transitions from extending in a first direction toward a first end (e.g., the proximal end 36 or the distal end 40) of the stent 30 at a bend and thereafter extends in a second direction toward the second end (e.g., the distal end 40 or the proximal end 36) of the stent 30. In other words, the turns 48 are points along the wire(s) where the wire bends back to extend in the opposite direction.

The segments 35 may have an inner surface and an outer surface, a thickness extending between the inner surface and the outer surface, and a length extending between turns 48 (e.g., between adjacent segments 35 or between a peak 39 and a valley 37 of adjacent segments 35). The thickness of the segments 35 may be uniform, but this is not required. In some instances, at least some of the segments 35 may vary in thickness and/or length.

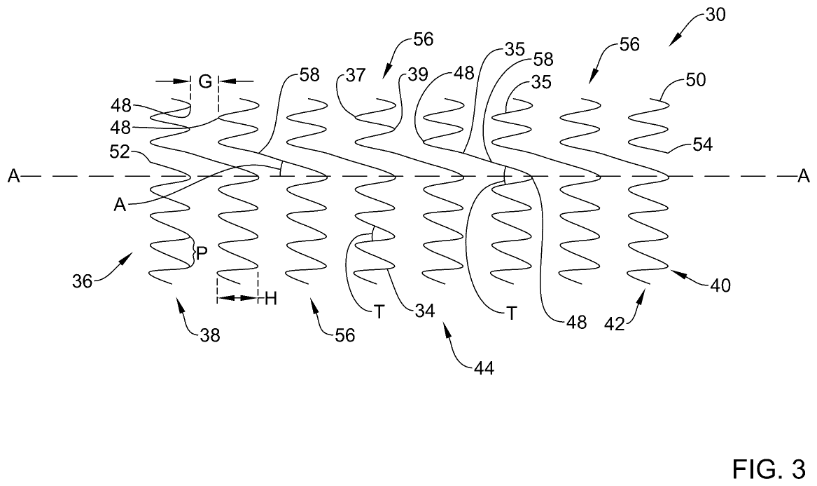

FIG. 3 depicts a schematic view of an illustrative configuration of the stent 30, where the framework 34 of the stent 30 has been split longitudinally (e.g., in the direction of line A-A) and laid flat from its intended tubular configuration with the annular rows 56 defining a lumen extending from the proximal end 36 to the distal end 40 (e.g., as described with respect to FIG. 2). The configuration of the stent 30 depicted in FIG. 3 may be formed from a single wire 50 having a first end 52 (e.g., a proximal end) and a second end 54 (e.g., a distal end), with the single wire 50 extending an entire length of the tubular framework 34.

The stent 30 may be a self-expanding stent (SES), meaning that the stent 30 may automatically expand into an expanded configuration (e.g., deployed configuration) once any constraints preventing expansion have been removed. In some instances, the stent 30 may not be a self-expanding stent, and thus may rely upon an inflatable balloon or other expandable structure to cause the stent 30 to expand from a collapsed configuration for delivery to its expanded configuration for deployment in a body lumen.

The wire 50 of the stent 30 may be formed from one or more suitable materials. Example suitable materials include, but are not limited to, metals, metal alloys, shape memory alloys, polymers, nickel-titanium alloys, cobalt-chromium-nickel-molybdenum alloys, and/or other suitable materials enabling the stent 30 to be expanded into a shape when positioned at a target site. In some instances, the material may be selected to enable the stent 30 to be removed with relative ease as well. In some examples, the wire 50 may be formed from alloys such as, but not limited to, nitinol and/or Elgiloy®.

The wire 50 may form a tubular framework 34 having a plurality of annular rows 56 (e.g., rows 56 may be configured to be annular when the framework 34 is in a tubular configuration) with each row 56 having a plurality of segments 35 and turns 48 between converging segments 35. The wire 50 may be configured to form the annular rows 56 of the stent 30 that define the framework 34 without the wire 50 overlapping itself and/or another wire along and/or between the rows 56 (e.g., with no segments 35 of the wire 50 overlap other segments 35 of the wire 50). In some examples, although the segments 35 of the wire 50 may not overlap other segments 35 of the wire 50, the segments 35 may have ends that are knotted or otherwise bent to form blunt ends.

Although the segments 35 may have a consistent length in each row 56 and among the different rows 56 (save for connecting segments 58) and the angle T of the turns 48 between segments 35 may be the same in each row 56 and among different rows 56, as depicted in FIG. 3, the segments 35 and/or the angles T of the turns 48 may differ within a row 56 and/or among the different rows 56. Although the angles T between the segments 35 that include a connecting segment 58 are depicted as being the same as angles T between adjacent segments 35 without a connecting segment 58, the angles T between segments 35 including the connecting segments 58 may be different from angles T between segments 35 that do not include connecting segments 58.

Each row 56 of the framework 34 formed from the wire 50 may have a longitudinal length H (e.g., a longitudinal distance between a valley 37 and a peak 39). The length H of each row 56 may be the same for each row 56, as depicted in FIG. 3, or a length H of one row 56 may be different than a length H of at least one other row 56.

Further, a longitudinal gap G may exist between each row 56, where the connecting segment 58 of the wire 50 may extend across the gap G between adjacent or consecutive rows 56. The gap G may extend circumferentially around the stent 30 between, for example, peaks of a first row 56 of the adjacent or consecutive rows 56 and valleys of a second row 56 of the adjacent or consecutive rows 56.

The gap G may have any suitable length or height between two closest turns 48 of adjacent rows 56. In some cases, a length of the gap G may be set based on a desired flexibility between rows 56 of the stent 30, but this is not required.

The connecting segments 58 of the wire 50 may be configured to span the gap G between adjacent or consecutive rows 56 and connect the adjacent or consecutive rows 56. In some cases, a single connecting segment 58 extending between two rows 56 may be the only portion of the wire 50 spanning the gap G and connecting adjacent or consecutive rows 56. Such a configuration may facilitate each row 56 providing suitable radial support to surrounding tissue, while providing flexibility to the stent 30 to allow the stent 30 to be used in a tortuous or non-straight duct due to the gap G providing a lower radial support than a radial support provided by the rows 56. In some cases, the single connecting segment 58 may be the only portion of the stent 30 that extends across or spans the gap G, but this is not required.

The connecting segments 58 may have any suitable length and may be set to any suitable angle A relative to a longitudinal line or axis A-A extending through turns 48 of the stent 30 (e.g., turns proximate a proximal end and/or distal end of the connecting segment 58). In some cases, the connecting segments 58 of the wire 50 may have a length greater than a distance of the sum of a length H of a first row 56 and a length H of an adjacent second row 56. For example, the connecting segments 58 of the wire 50 may have a length of at least a distance of the sum of a length H of a first row 56, a length H of an adjacent second row 56, and a distance of the gap G between the first row 56 and the adjacent second row 56. More specifically, in some examples, a length of the connecting segment 58 may be equal to the sum of a length H of a first row 56, a length H of an adjacent second row 56, and a distance of the gap G, where the sum is divided by the cosine of angle A.

Angle A may be any suitable angle between the line or axis A-A. In some examples, the angle A may be an oblique angle. In some examples, the angle A may be a value between zero degrees and sixty degrees, but other suitable values are contemplated.

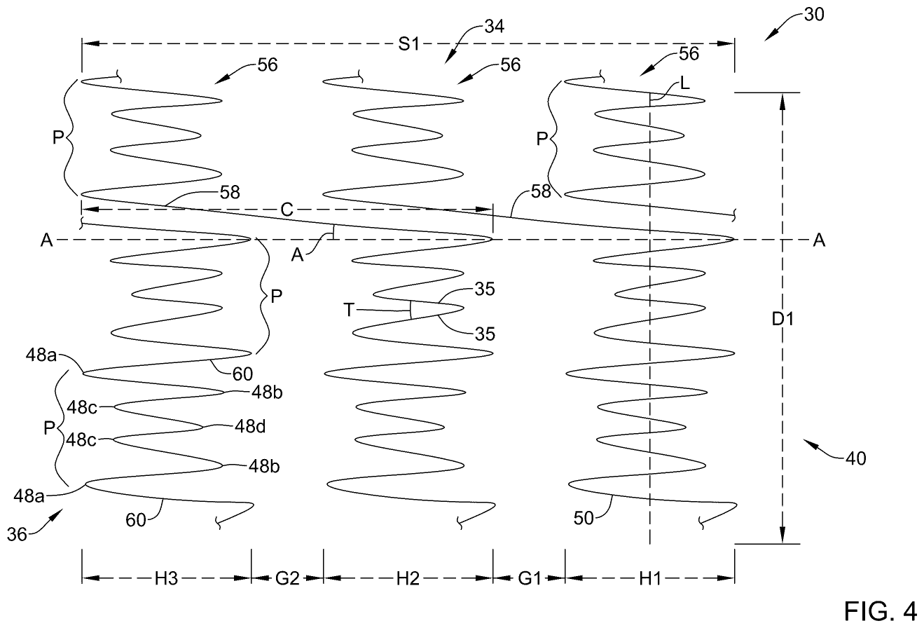

FIG. 4 is a schematic view of a section of framework 34 of an illustrative configuration of the stent 30, where the segments 35 in each row 56 form a configuration of a pattern P that repeats along the row 56 and framework 34 is laid flat. In some examples and as depicted in FIG. 4, adjacent iterations of the pattern P face opposite directions, but this is not required. In some cases, the two adjacent iterations of the pattern P facing opposite directions may be considered to form a further configuration of a pattern.

The pattern P may have any suitable configuration comprising any suitable number of segments 35 having an angle T between adjacent segments (e.g., where the angle T may be the same or differ along the stent 30). The segments 35 may have any suitable length, which may be based on locations of the turns 48 and/or other suitable factors. Angle T may be any suitable angle between adjacent segments 35. In some examples, the angle T may be a value between zero degrees and sixty degrees, but other suitable values are contemplated. In some examples, the pattern P may include two segments of equal length that repeat along a row 56 between connecting segments, for example as depicted in FIG. 3. In some examples, the pattern P may include six segments 35, where adjacent iterations of the pattern P may be connected by an intermediate segment 60, as depicted in FIG. 4. In such configurations including the intermediate segments 60, the turns 48 of two consecutive iterations of the pattern P may be independent of one another.

The configuration of the pattern P may be described by the number of turns 48 included in the pattern (e.g., a number of partial turns (e.g., with a single segment 35 extending therefrom) or full turns (e.g., with two segments 35 extending therefrom) and the pattern P may have any suitable number of turns 48. In some examples, the pattern P may have at least three turns 48, where each of the three turns 48 is at a different axial location along the row 56, as depicted in FIG. 4. Additionally or alternatively, in some examples, the pattern P may include at least two turns 48 at a same axial location along the row 56, as depicted in FIG. 4. When the pattern P includes two turns 48 at a same axial location, those turns 48 may be independent of turns 48 of an adjacent instance of the pattern P of turns 48 and segments 35.

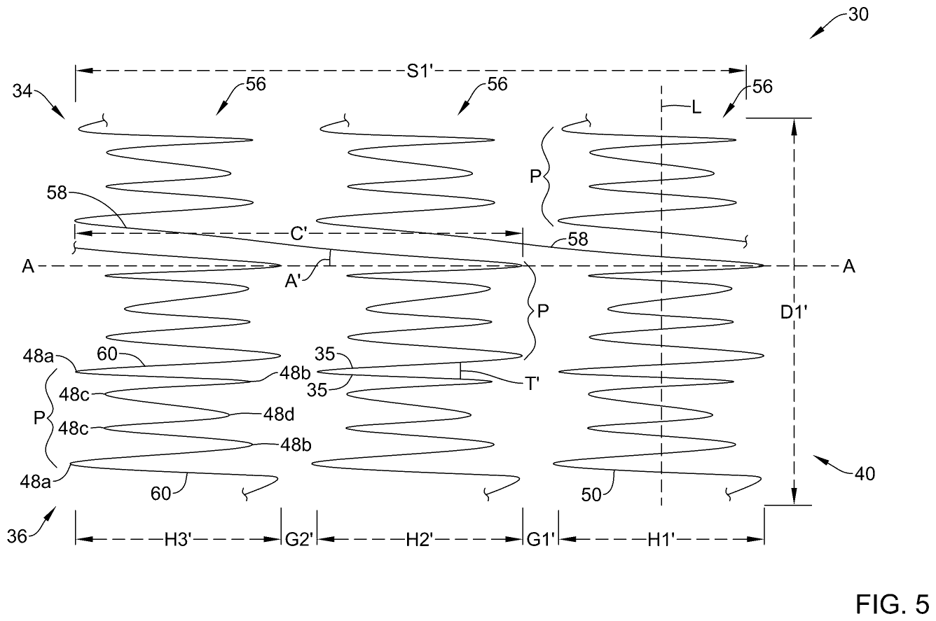

The configuration of the pattern P depicted in FIG. 4 may include seven turns 48. In some examples, the seven turns 48 may include two laterally outward-most turns 48a at a same axial location, a next two laterally inward turns 48b at a different axial location than the two laterally outward-most turns 48a, a further next two laterally inward turns 48c at a different axial location the turns 48a, 48b, and a center turn 48d at a different axial location than the two laterally outward-most turns 48a, the next two laterally inward turns 48b, and the further next two laterally inward turns 48c. The pattern P depicted in FIG. 4, along with the connecting segment 58 extending between and connecting adjacent rows 56 may facilitate preventing and/or mitigating foreshortening of the stent 30 as the stent adjusts between a constrained configuration (e.g., in a delivery configuration, as depicted in FIG. 5) and a relaxed or unconstrained (e.g., a deployed configuration, as depicted in FIG. 4).

Any suitable iterations of the pattern P may be provided in each row 56 of the stent 30 and the number of iterations of the pattern P in a row 56 may be the same as the number of iterations in all or one or more other rows or different than all or one more other rows 56. In some examples, the pattern P may repeat a same number of times in each row 56 and/or may repeat three times per row 56, repeat four times per row 56, repeat six times per row 56, repeat eight times per row 56, and/or repeat one or more other suitable times per row 56.

Decreasing or increasing the number of times the pattern P may repeat in a row 56 while maintaining a diameter of the stent 30 may lower or increase, respectively, an amount of radial force the stent 30 may be configured to apply to surrounding tissue when deployed. Decreasing or increasing the number of times the pattern P may repeat in a row 56 may require scaling the pattern P up or down, respectively, such that scaling the size of the pattern P may have a direct impact on the amount of radial force the stent 30 is configured to apply to surrounding tissue when deployed.

The iterations of the pattern P may be equally or unequally circumferentially spaced around a row 56. Spacing of the iterations of the pattern P may be adjusted to control an amount of and/or a location of radial force applied by the stent 30 to surrounding tissue. In some examples, the iterations of the pattern P may be equally or substantially equally circumferentially spaced around the row 56.

The rows 56 may be centered about a line L-L at an axial location along a length of the rows 56 of the stent 30, which may be equidistance between the farthest longitudinally spaced turns 48 of the row 56. In some examples, a height between all sets or at least one set of the peaks 37 and valleys 39 (e.g., where a set of valleys 37 and peaks 39 may be adjacent valleys 37 and peaks 39, the farther apart valley 37 and peak 39, the closest valley 37 and peak 39, etc.) of a row 56 may be centered about the line L-L.

The line L-L may be at any suitable angle relative to a longitudinal axis or line A-A of the stent 30. In some examples, the line L-L for all rows 56 or one or more rows 56 may be perpendicular to the longitudinal axis and/or the line A-A of the stent 30, as depicted in FIG. 4. In some examples, the line L-L for all rows 56 or one or more rows 56 may be non-perpendicular to the longitudinal axis and/or the line A-A of the stent 30 (e.g., such that the rows 56 follow a helical pattern and/or other suitable pattern along a length of the stent).

The section of the framework 34 depicted in FIG. 4 is shown in a relaxed or unconstrained configuration (e.g., a deployed configuration) having the dimensions shown and described below. The three rows 56 of the framework 34 that are depicted have lengths of H1, H2, and H3, respectively, with gaps having a length G1 and a length G2 between adjacent rows 56. In some cases, the lengths H1, H2, and H3 may all be equal or substantially equal and the lengths G1 and G2 may be equal or substantially equal. A length S1 of the section of the framework 34 depicted in FIG. 4 may be equal to or substantially equal to the sum of H1, H2, H3, G1, and G2. Each section of the rows 56 depicted in FIG. 4 may have a length equal to D1. The connecting segments 58 may have a length C.

FIG. 5 depicts the section of the framework 34 of the illustrative configuration of the stent 30 depicted in FIG. 4, where the framework 34 is in a compressed or constrained configuration or state (e.g., a delivery configuration). Due to constraining the stent 30, a diameter of the stent 30 may be reduced relative to a relaxed or unconstrained stent 30, such that certain dimension of stent 30 may change relative to when the stent 30 is unconstrained while other dimensions of the stent 30 may remain unchanged. The dimensions depicted in FIG. 4 are reproduced in FIG. 5 with a “′” thereafter to indicate the depicted dimensions refer to the sizes when the stent 30 is in a constrained configuration relative to the unconstrained configuration depicted in FIG. 4.

The reduced diameter of the stent 30 when in the constrained configuration may be represented by the length D1′ of the section of the rows 56 depicted in FIG. 5, where the length D1′ may be less than the length D1. Although an angle T′ is reduced relative to the angle T between adjacent segments 35, the lengths H1′, H2′, H3′ of the rows 56 are longer than the lengths H1, H2, H3 of the rows 56, and the length G1′ and G2′ of the gaps between adjacent rows 56 are smaller than the lengths G1 and G2, the total length S1′ of the section of the framework 34 depicted in the constrained configuration of FIG. 5 is equal to or substantially equal to the total length S1 of the section of framework 34 depicted in the relaxed or unconstrained configuration of FIG. 4. Similarly, the length C′ of the connecting segment 58 and angle A′ between the connecting segment 58 and the line or axis A-A in the constrained configuration of the stent 30 may be, respectively, equal to or substantially equal to the length C of the connecting segment 58 and the angle A between the connecting segment and the line or axis A-A in the unconstrained or relaxed configuration of the stent 30. As a result, the stent 30 including the framework 34 depicted in FIGS. 4 and 5 may maintain its length when the stent 30 adjusts from a constrained configuration to an unconstrained configuration (e.g., there is no or substantially no foreshortening of the stent 30), while outputting a desired radial force on tissue proximate the stent and having a desired flexibility along the length of the stent 30.

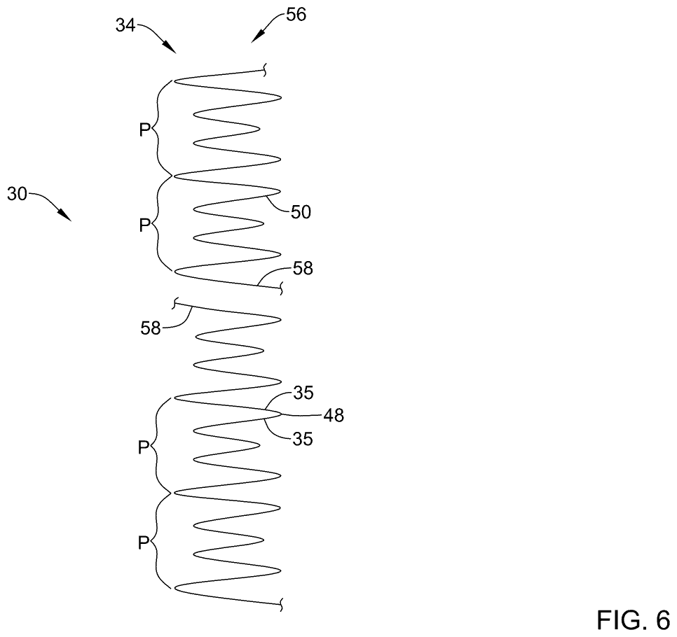

FIG. 6 depicts a schematic view of a portion of a row 56 of a section of the framework 34 of an illustrative configuration of the stent 30, where the row 56 of the framework 34 has been longitudinally split and laid flat. Further, the wire 50 of the framework 34 depicted in FIG. 6 is in a relaxed or unconstrained (e.g., deployed) configuration.

The configuration of the wire 50 of the stent 30 depicted in FIG. 6 includes the wire 50 in the pattern P discussed above with respect to FIGS. 4 and 5, where the pattern P is oriented in a same direction. As the pattern P of the wire 50 is oriented in the same direction, the use of intermediate segments 60 between adjacent patterns P may be omitted, such that consecutive iterations of the pattern P share a turn 48. Each row 56 of the wire 50 of the stent 30 may begin and end with separate connecting segments 58, as depicted in FIG. 6, configured to connect the row 56 with adjacent rows of the stent 30.

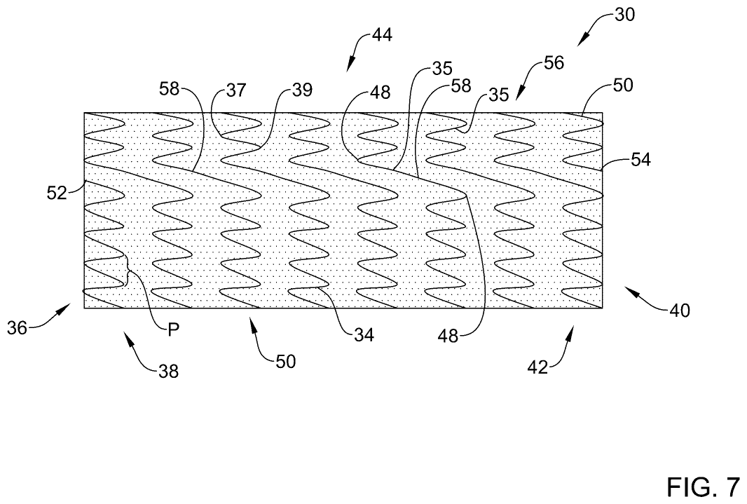

FIG. 7 depicts the illustrative configuration of the framework 34 of the stent 30 depicted in and discussed with respect to FIG. 3, where the stent 30 includes a coating 62 (e.g., represented by a dotted layer) applied to the framework 34 (e.g., applied to one or more of the rows 56 of the framework 34). In some cases, one or more layers of the coatings 62 may be applied to the framework 34 (e.g., the formed framework 34). For example, one or more layers of material may be applied to an inner surface of the framework 34 and/or one or more layers of material may be applied to an outer surface of the framework 34.

The coating 62, when applied to the framework 34, may be applied to any suitable portion of the framework 34. As depicted in FIG. 7, the coating 62 may be applied to an entirety of the framework 34, but this is not required and the coating 62 may be applied to only a portion of the framework 34 that is less than the entirety of the framework 34. The coating 62 may be a sheet of material and/or may take on one or more other suitable forms.

The coating 62 may be formed from any suitable material. In some examples, the coating 62 may be formed from a polymeric material, metallic material, a fabric material, a liquid material, and/or other suitable materials. In some examples, the coating 62 may be configured to prevent and/or mitigate tissue ingrowth into the stent 30 such that the stent 30 may be more easily removed from a target site. In one example, the coating 62 may be a polymeric material configured to mitigate and/or prevent tissue ingrowth.

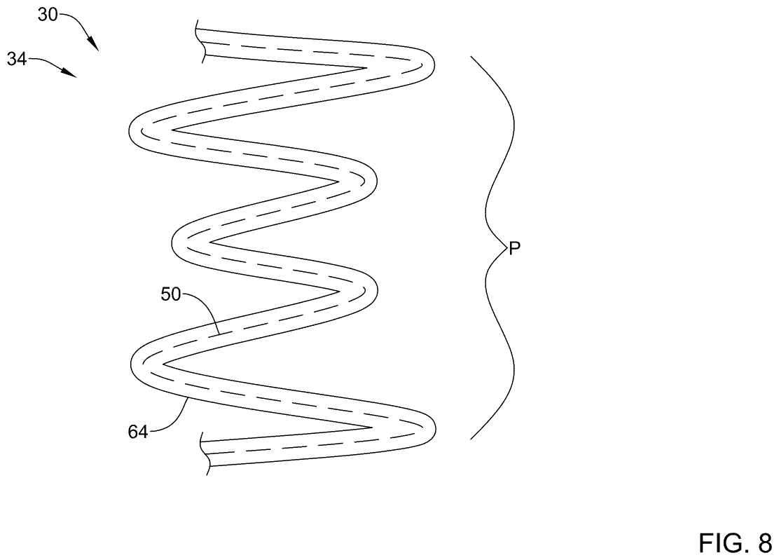

FIG. 8 depicts an illustrative portion of the wire 50 formed in the pattern P, where a coating 64 is applied to and/or around the wire 50. The coating 64, when applied to the wire 50, may be applied to any suitable portion of the wire 50. As depicted in FIG. 8, the coating 64 may be applied around an entirety of the wire 50, but this is not required and the coating 62 may be applied to only one or more portions of a circumference of the wire 50 and/or one or more portions of a length of the wire 50. Although not required, the wire 50 may be dipped in a coating before or after being formed into the framework 34 to form the coating 64 on the wire 50.

The coating 64 may be formed from any suitable material. In some examples, the coating 64 may be formed from a polymeric material, a metallic material, and/or other suitable materials. In some examples, the coating 62 may be configured to prevent and/or mitigate tissue ingrowth into the stent 30 such that the stent 30 may be more easily removed from a target site and/or to set a radial force of the stent 30. In one example, the coating 62 may be a polymeric material configured at least to mitigate and/or prevent tissue ingrowth. Some examples of suitable materials for the stents 30 and/or coating 64 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

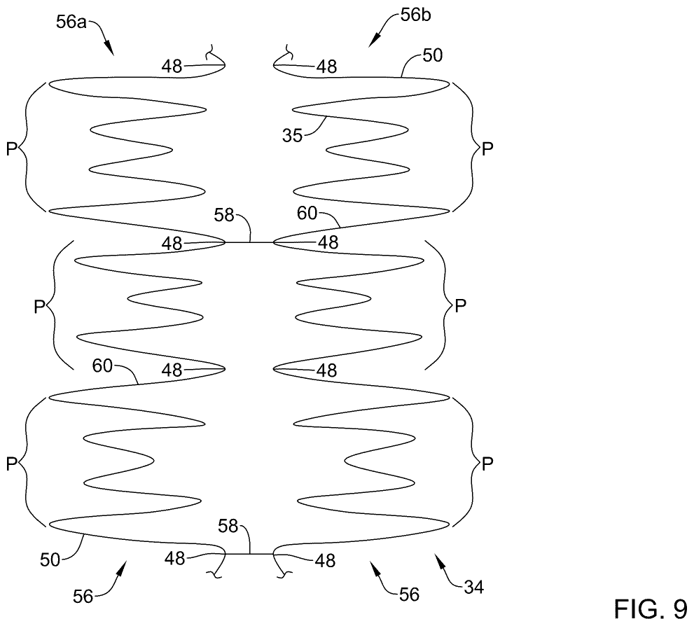

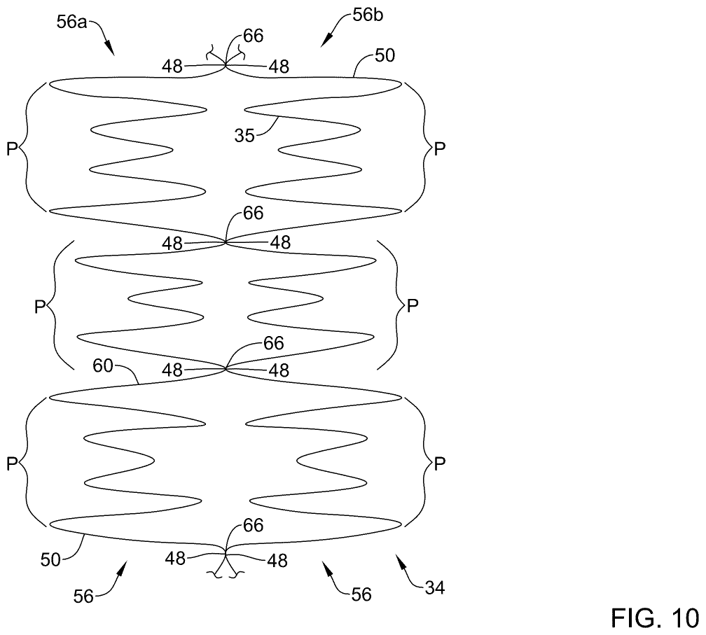

In some cases, portions of the stent 30 may be modularly formed and coupled to one another to allow for customized lengths, customized radial forces the stent 30 may be configured to apply to tissue, customized flexibility and/or rigidity of the stent 30, customized parameters (e.g., lengths, radial forces, flexibility, rigidity, etc.) along a length or circumference of the stent 30, and/or to allow for other suitable customizations. FIGS. 9 and 10 depict sections of rows 56 of the framework of the stent 30 that have been modularly formed and coupled to one another.

FIG. 9 depicts a schematic view of portions of adjacent rows 56 of a section of the framework 34 of an illustrative configuration of the stent 30, where the rows 56 of the framework 34 have been longitudinally split and laid flat. Further, the wire 50 of the framework 34 depicted in FIG. 9 is in a relaxed or unconstrained (e.g., deployed) configuration.

The configuration of the wire 50 of the stent 30 depicted in FIG. 9 includes the wire 50 in the configuration of the pattern P discussed above with respect to FIG. 6 (e.g., with the pattern P within a row 56 oriented in a same direction). However, the patterns P in adjacent rows 56 are mirror images or face in opposite directions. For example, the pattern P in a first row 56a may face an opposite direction relative to a direction the pattern P in a second row 56b faces.

The adjacent rows 56 in the illustrative configuration of the framework 34 depicted in FIG. 9 may be coupled to one another via the connecting segment 58, where the connecting segment 58 may be a section of wire (e.g., the wire 50 forming the pattern P and/or other suitable wire). In some examples, one or more turns 48 between the first row 56a may be coupled to one or more turns 48 of the second row 56b with the connecting segment 58 extending therebetween.

Any suitable number of connecting segments 58 may extend between the modularly formed rows 56. In some examples, the connecting segment 58 may be applied between adjacent rows 56 at each set of closest turns 48 of the respective rows 56a, 56b, at intervals around a circumference of the rows 56a, 56b, and/or at a single set of closest turns 48 of the respective rows 56a, 56b. In one example, the connecting segment 58 may be applied between adjacent rows 56 every other (e.g., every third) set of closest turns 48, as depicted in FIG. 9, but this is not required.

The connecting segments 58 extending between sets of closest turns 48 of adjacent rows 56 of the framework 34 may be coupled to the wire 50 in any suitable manner. Example suitable manners for coupling the connecting segments 58 to the wire 50 include, but are not limited to, welding the connecting segments 58 to the wire 50, tying the connecting segments 58 to the wire 50, and/or coupling the connecting segments 58 to the wire 50 in one or more other suitable manners.

FIG. 10 depicts a schematic view of portions of adjacent annular rows 56 of a section of the framework 34 of an illustrative configuration of the stent 30, where the annular row 56 of the framework 34 has been longitudinally split and laid flat. Further, the wire 50 of the framework 34 depicted in FIG. 10 is in a relaxed or unconstrained (e.g., deployed) configuration.

The configuration of the wire 50 of the stent 30 depicted in FIG. 10 includes the wire 50 in the configuration of pattern P discussed above with respect to FIGS. 4 and 5 (e.g., with the pattern P within a row 56, with every other iteration of the pattern P oriented in an opposite direction). In other words, the pattern P alternates in opposite directions. However, the patterns P in adjacent rows 56 are mirror images or face in opposite directions. For example, the pattern P in a first row 56a may face an opposite direction relative to a direction the pattern P in a second row 56b faces.

The adjacent rows 56 in the illustrative configuration of the framework 34 depicted in FIG. 10 may be directly coupled to one another. In some examples, one or more turns 48 between the first row 56a may be directly coupled to one or more turns 48 of the second row 56b at respective coupling points 66.

Any suitable number of coupling points 66 may be utilized to couple the modularly formed rows 56. In some examples, the coupling points 66 may be applied between adjacent rows 56 at each set of closest turns 48 of the respective rows 56a, 56b, at spaced intervals around a circumference of the rows 56a, 56b, and/or at a single set of closest turns 48 of the respective rows 56a, 56b. In one example, the coupling points 66 may be applied between adjacent rows 56 every other (e.g., every third) set of closest turns 48, for example as depicted in FIG. 10, but this is not required.

The coupling points 66 between sets of closest turns 48 of adjacent rows 56 of the framework 34 may be formed by coupling the respective closest turns 48 of adjacent rows 56 to one another in any suitable manner. Example suitable manners for coupling the closest turns 48 of adjacent rows 56 to one another include, but are not limited to, welding the closest turns 48 of adjacent rows 56 to one another, tying the closest turns 48 of adjacent rows 56 to one another, and/or coupling the closest turns 48 of adjacent rows 56 to one another in one or more other suitable manners. In one example, the closest turns 48 of adjacent rows 56 may be welded to one another to form the coupling points 66.

The framework 34 of the stent 30 may be formed in any suitable manner. In some examples, the framework 34 may be formed by wrapping the wire 50 around pins in and extending radially outward from one or more mandrels to form the turns 48 of a desired pattern. Once a desired configuration of the framework 34 is formed, the framework may be heat treated to cause the wire to self-expand to the desired configuration of the framework 34 when it is adjusted from a constrained configuration and unconstrained configuration, but this is not required. Other suitable configurations and/or techniques for forming the framework 34 of the stent 30 are contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent, comprising:
a tubular member having a first end, a second end, and a lumen between the first end and the second end, and
wherein a wire having a first end and a second end forms a plurality of annular rows between the first end and the second end that define the lumen,
wherein the wire has no overlapping portions along the plurality of rows,
wherein the wire forms a plurality of turns in each row of the plurality of rows,
wherein a gap extends between consecutive rows of the plurality of rows, and
wherein the wire includes a connecting segment that spans across the gap to interconnect two rows of the plurality of rows.

2. The stent of claim 1, wherein the connecting segment is the only portion of the wire that spans across the gap.

3. The stent of claim 1, wherein the connecting segment is at an oblique angle relative to a longitudinal axis of the plurality of rows.

4. The stent of claim 1, wherein:
a first row of the consecutive rows has a first height and a second row of the consecutive rows has a second height, and
the connecting segment has a length greater than a sum of the first height and the second height.

5. The stent of claim 1, wherein:
the plurality of turns in each row define peaks at a first axial end of a row and valleys at a second axial end of a row, and
the gap circumferentially extends between all of the peaks of a first row of the consecutive rows and all of the valleys of a second row of the consecutive rows.

6. The stent of claim 1, wherein:
the plurality of turns in each row define peaks at a first axial end of a row and valleys at a second axial end of a row, and
a height between the peaks and valleys of at least one row of the plurality of rows is centered about an axial location along a length of the plurality of rows.

7. The stent of claim 1, wherein the plurality of turns forms a pattern that repeats in a row of the plurality of rows and the pattern comprises three turns with each of the three turns at a different axial location of the row of the plurality of rows.

8. The stent of claim 1, wherein the plurality of turns forms a pattern that repeats in a row of the plurality of rows and the pattern comprises two turns with each of the two turns at a same axial location of the row of the plurality of rows.

9. The stent of claim 8, wherein the two turns at the same axial location are independent of turns of an adjacent instance of the pattern.

10. The stent of claim 1, wherein a coating is applied to the plurality of rows.

11. A stent, comprising:
a wire defining a tubular member with a lumen, the wire having a first end and a second end, and
wherein the wire defines a plurality of annular rows between the first end and the second end, and
wherein each row has a pattern that repeats, and the pattern includes three or more turns with each of the three or more turns at a different axial location along a length of the plurality of rows and two or more turns with each of the two or more turns at a same axial location along the length.

12. The stent of claim 11, wherein the two or more turns at the same axial location along the length are independent of turns in an adjacent instance of the pattern.

13. The stent of claim 11, wherein the wire has no segments that overlap other segments of the wire.

14. The stent of claim 11, wherein each row is centered about an axial location along the length of the plurality of rows.

15. The stent of claim 11, wherein a single connecting segment of the wire connects consecutive rows of the plurality of rows.

16. A stent, comprising:
a wire defining a tubular member with a lumen, the wire having a first end and a second end, and
wherein the wire defines a plurality of annular rows between the first end and the second end, and
wherein each row has a pattern that repeats circumferentially, and the pattern includes two or more turns each at a same axial location along a length of the plurality of rows and all turns of the pattern are independent of all turns in an adjacent instance of the pattern.

17. The stent of claim 16, wherein the pattern includes three or more turns each at a different axial location along the length of the plurality of rows.

18. The stent of claim 16, wherein the wire has no segments that overlap other segments of the wire.

19. The stent of claim 16, wherein each row is centered about an axial location along the length of the plurality of rows.

20. The stent of claim 16, wherein a single connecting segment of the wire connects consecutive rows of the plurality of rows.
